(19) United States
(12) Patent Application Publication
(10) Pub. No.: US 2025/0249139 A1
(43) Pub. Date: Aug. 7, 2025

(54) ESSENTIAL OIL DIFFUSER

(71) Applicant: SCENTSANEE DIFFUSING INC., MISSISSAUGA (CA)

(72) Inventor: SANDEEP PABLA, Mississauga (CA)

(21) Appl. No.: 19/044,567

(22) Filed: Feb. 3, 2025

Related U.S. Application Data
(60) Provisional application No. 63/549,579, filed on Feb. 5, 2024.

(51) Int. Cl. A61L 9/14

(52) U.S. Cl. CPC A61L 9/14; A61L 2209/11; A61L 2209/13; A61L 2209/133

(57) ABSTRACT

An essential oil diffuser comprising a top housing and a bottom housing is disclosed. The bottom housing includes two or more vial receiving areas configured for receiving two or more vials. The two or more vials store essential oils. The bottom housing includes a water chamber. At least one actuator operates the vial to dispense essential oil into the water chamber. The essential oil diffuser comprises a transducer that vibrates such that small droplets of water and the essential oil are released into the air as a cool mist. In one example, the essential oil diffuser communicatively connects to an electronic device. The electronic device remotely operates the essential oil diffuser to dispense the essential oils from one or more vials into the water chamber.

### CROSS-REFERENCE TO RELATED APPLICATIONS

This application claims priority to U.S. Provisional Patent Application No. 63/549,579, titled “AN ESSENTIAL OIL DIFFUSER” filed Feb. 5, 2024, which is hereby incorporated by reference in its entirety.

### FIELD OF INVENTION

The present subject matter generally relates to oil diffusers. More specifically, the present subject matter relates to an essential oil diffuser configured to store multiple essential oils and dispense them both manually and electronically into an atomizing chamber.

### BACKGROUND OF INVENTION

An oil diffuser is a device configured to disperse an essential oil into the air. The essential oil offers a pleasant aroma, promotes relaxation, and potentially provides health benefits through the inhalation of the essential oils. The oil diffuser is also called as an essential oil diffuser. The oil diffusers come in a variety shapes and forms. One example is disclosed in a United States granted U.S. Pat. No. 10,034,987, entitled “Essential oils diffuser” (“the '987 Patent”). The '987 Patent discloses an essential oils diffuser with a micro air pump providing between 500 and 1500 Pa of pressure with a consumption of between 0.2 and 1 Watts may be used to push air into essential oils or an essential oil solution to diffuse and expel the oils into the air, while keeping the oils away from the air pump, preventing the oils from shortening the life of the diffuser. The diffuser may include a power supply and a controller to regulate the operation of the diffuser.

Another example is disclosed in a United States Publication no. 20070242464, entitled “Essential oil diffuser” (“the '464 Publication”). The '464 Publication discloses a diffuser assembly comprising a top cap having an opening, a diffusing member having a housing tip which is protruded out the opening of the top cap in order to prevent an essential oil from leaking out an inner surface of the top cap, an O-ring fastened on a bottom of the diffusing member, an air pump having a outlet for blowing out an air, a base, a flexible pipe attached to the outlet of the air pump and fixed on one top surface of the base, an oil reservoir having a holding bar formed on a bottom of the oil reservoir, wherein there exists a slender hole inside the holding bar so that the air pump is activated to let the air be supplied to the oil reservoir attached by the flexible pipe through the outlet of the air pump and the holding bar, a plurality of lighting elements disposed on the top surface of the base for emitting rays on the top cap and the oil reservoir in order to obtain good visual effects, a foam surrounding the air pump for absorbing a vibration and a noise, a mat disposed on a bottom of the air pump for absorbing a vibration and a noise, and a lower lid closing the bottom of the base, wherein the foam and the air pump are affixed on the lower lid and the diffusing member is sealed with the opening of the oil reservoir through the O-ring.

Although the above discussed oil diffusers are useful, they have few problems. For instance, the vast majority of existing essential oil diffusers function in a similar manner i.e., they have a manual button to start and stop the diffuser once the essential oil is added. With recent improvements in technology, the essential oil diffusers have been integrated with smartphones to control the oil diffuser basic functions such as turning ON/OFF remotely. A person skilled in the art understands that manually turning on the diffuser and manually adding drops of oil into the diffuser are both time-consuming. Further, many times the user adds too many drops by accident using up the oil too fast over time and/or requires many oils to be added into the water together to take advantage of multiple benefits simultaneously only to find themselves searching or spending time gathering the oils needed for the recipe, again time-consuming and many times a deterrent to use or benefiting fully from diffusing. Further, only one oil can be added at a time. Furthermore, the users may not have a recipe handy to know which oils to add to obtain a particular health benefit i.e., to create a “focus blend”.

Therefore, there is a need in the art to provide an improved essential oil diffuser that allows the user to store two or more vials or containers, each containing an essential oil, and to eject drops of those oils from each of the vials into a water basin with ease.

### SUMMARY OF THE INVENTION

It is an object of the present subject matter to provide an essential oil diffuser with multiple vial receiving areas, actuator-controlled dispensing, and mist generation capabilities.

It is another object to provide an essential oil diffuser with a plunger-based vial design, dual-extension actuators, and wireless control features.

In an exemplary embodiment, an essential oil diffuser is described. The essential oil diffuser comprises a bottom housing and a top housing. The bottom housing comprises a base having upstanding cylindrical walls forming a water chamber, a plurality of vial receiving areas, and a transducer disposed in the water chamber. Each vial receiving area is configured to receive a vial containing an essential oil in an inverted orientation. Each vial receiving area comprises an oil dispensing port opening into the water chamber. The top housing is mounted on the bottom housing. The top housing comprises at least one actuator positioned to engage with at least one vial when received in a respective vial receiving area. The at least one actuator is configured to apply a force on the at least one vial to dispense essential oil through the oil dispensing port into the water chamber. The transducer is configured to generate a mist from a mixture of the essential oil and water in the water chamber. A vent opening is configured to release the generated mist.

In some embodiments, each vial comprises a glass bottle for storing the essential oil, a cylindrical casing, a plunger assembly movably disposed within the cylindrical casing, a spring and a ball positioned below the spring. The ball functions as a check valve to enable unidirectional oil flow during dispensing operations.

In some embodiments, the at least one actuator comprises a first extension and a second extension configured to independently engage with two vials when received in respective vial receiving areas.

In some embodiments, the essential oil diffuser further comprises a controller and a transceiver electronically connected to the controller. The transceiver is configured to communicate with an electronic device over a network to receive control signals for operating the at least one actuator.

In some embodiments, the controller is configured to monitor an amount of essential oil remaining in each vial and transmit, via the transceiver, a notification to the electronic device when the amount of essential oil in any vial falls below a predetermined threshold.

In some embodiments, the controller is configured to activate the actuator according to a predetermined schedule received from the electronic device.

In some embodiments, the plurality of vial receiving areas comprises four vial receiving areas arranged in the bottom housing.

In some embodiments, each vial is configured to store the essential oil and is calibrated to dispense the essential oil in increments of a predetermined volume.

In some embodiments, the essential oil diffuser further comprises electrical strips disposed on an inner side of the top housing and configured to supply current to the at least one actuator.

In some embodiments, each vial is configured to be removable from the vial receiving area, refillable with the same or different essential oils when removed, interchangeable with other vials containing different essential oils, and reusable with a pump nozzle head for topical application.

In some embodiments, the bottom housing comprises an inner cover. The inner cover is a domed wall portion extending upward from the upstanding cylindrical walls and converging toward the vent opening of the top housing. The domed wall portion includes an interior surface configured to direct the generated mist from the water chamber toward the vent opening.

In some embodiments, the controller is configured to receive, via the transceiver, a blend recipe comprising instructions for dispensing at least one essential oil from at least one vial and activate at least one actuator according to the blend recipe to dispense a mixture of essential oils in the water chamber.

In some embodiments, the at least one actuator is configured to provide controlled dispensing through multiple discrete actuations. Each actuation of the actuator creates a single dispensing operation that releases a precise, predetermined volume of essential oil. In some embodiments, the controller is further configured to store usage patterns of different essential oil combinations, store the blend recipe for subsequent use, and generate and transmit recommended blend recipes to the electronic device based on the stored usage patterns.

In some embodiments, the controller is further configured to maintain a usage log for each vial received in the vial receiving areas, calculate an estimated remaining usage volume for each vial based on the respective usage log and a predetermined volume capacity, and automatically transmit, via the transceiver to the electronic device, a reordering notification when the estimated remaining usage volume falls below a predetermined threshold.

In another exemplary embodiment, an essential oil diffuser is described. The essential oil diffuser comprises the structural elements of the first embodiment with additional controller functionality. The controller is electronically connected to the at least one actuator and the transducer. A transceiver is electronically connected to the controller and configured to communicate with an electronic device over a wireless network. A memory stores executable instructions that, when chamber and the controller, cause the controller to receive blend recipes from the electronic device, activate the at least one actuator according to the blend recipes to create customized mixtures of essential oils in the water chamber, and monitor amounts of essential oils remaining in the vials and transmit notifications to the electronic device.

In yet another exemplary embodiment, a method of operating an essential oil diffuser is described. The method comprises receiving a plurality of vials in respective vial receiving areas of a bottom housing in an inverted orientation. Each vial contains an essential oil. Each vial receiving area comprises an oil dispensing port opening into a water chamber formed by upstanding cylindrical walls of the bottom housing. The method further comprises filling the water chamber with water to a predetermined level, mounting a top housing on the bottom housing. The top housing includes at least one actuator positioned above at least one of the received vials. The method comprises activating the at least one actuator to apply a downward force on the at least one received vial, thereby causing essential oil to be dispensed through the oil dispensing port into the water chamber. The method includes operating a transducer disposed in the water chamber to generate a mist from a mixture of the dispensed essential oil and the water and releasing the generated mist through a vent opening in the top housing.

The features and advantages described here will become more apparent through the detailed description of selected embodiments illustrated in the accompanying FIGURES. The subject matter is capable of modifications without departing from its scope. The drawings and description should be regarded as illustrative rather than limiting.

### BRIEF DESCRIPTION OF THE DRAWINGS

Further features and advantages of the present subject matter will become apparent from the following detailed description, taken in combination with the appended drawings, in which:

FIG. 1 illustrates a perspective view of an essential oil diffuser, in accordance with one exemplary embodiment of the present subject matter;

FIG. 2A illustrates an exploded view of the essential oil diffuser, in accordance with one exemplary embodiment of the present subject matter;

FIG. 2B illustrates another exploded bottom view of the essential oil diffuser, in accordance with one exemplary embodiment of the present subject matter;

FIG. 3A illustrates an exploded view of the bottom housing, in accordance with one exemplary embodiment of the present subject matter.

FIG. 3B illustrates a perspective view of the bottom housing, in accordance with one exemplary embodiment of the present subject matter;

FIG. 4 illustrates the bottom housing with at least one actuator engaged with the vials, in accordance with one exemplary embodiment of the present subject matter;

FIG. 5 illustrates a top view of the bottom housing, in accordance with one exemplary embodiment of the present subject matter;

FIG. 6 illustrates an exploded view of vial, in accordance with one exemplary embodiment of the present subject matter;

FIG. 7A illustrates a front view of vial, in accordance with another embodiment of the present subject matter;

FIG. 7B illustrates a cross-section view of vial, in accordance with another embodiment of the present subject matter; and

FIG. 8 illustrates an environment of the essential oil diffuser communicatively connected to an electronic device for remotely operating the essential oil diffuser, in accordance with one exemplary embodiment of the present subject matter.

It will be noted that throughout the appended drawings, like features are identified by like reference numerals.

### DETAILED DESCRIPTION OF THE EMBODIMENTS

The following detailed description set forth below in connection with the appended drawings is intended as a description of exemplary embodiments in which the presently disclosed subject matter may be practiced. The term “exemplary” used throughout this description means “serving as an example, instance, or illustration,” and should not necessarily be construed as preferred or advantageous over other embodiments. The detailed description includes specific details for providing a thorough understanding of the presently disclosed an essential oil diffuser. However, it will be apparent to those skilled in the art that the presently disclosed subject matter may be practiced without these specific details. In some instances, well-known structures and devices are shown in functional or conceptual diagram form in order to avoid obscuring the concepts of the presently disclosed the essential oil diffuser.

In the present specification, an embodiment showing a singular component should not be considered limiting. Rather, the subject matter preferably encompasses other embodiments including a plurality of the same component, and vice-versa, unless explicitly stated otherwise herein. Moreover, the applicant does not intend for any term in the specification to be ascribed an uncommon or special meaning unless explicitly set forth as such. Further, the present subject matter encompasses present and future known equivalents to the known components referred to herein by way of illustration.

Although the present subject matter provides a description of the essential oil diffuser, it is to be further understood that numerous changes may arise in the details of the embodiments of the essential oil diffuser. It is contemplated that all such changes and additional embodiments are within the spirit and true scope of this disclosure.

The following detailed description is merely exemplary in nature and is not intended to limit the described embodiments or the application and uses of the described embodiments. All of the implementations described below are exemplary implementations provided to enable persons skilled in the art to make or use the embodiments of the disclosure and are not intended to limit the scope of the disclosure.

Various features and embodiments of the essential oil diffuser are explained in conjunction with the description of FIGUREs (FIGS.) 1-8.

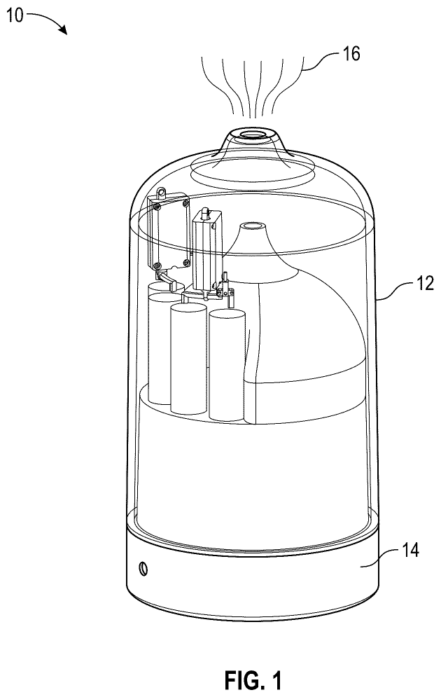

FIG. 1 illustrates a perspective view of an essential oil diffuser 10, in accordance with one exemplary embodiment of the present subject matter. The essential oil diffuser 10 includes a top housing 12 and a bottom housing 14 and is configured to release a generated mist 16 through a vent opening into the surrounding environment. The essential oil diffuser 10 further comprises an inner cover 28. The outer housing 12 forms the overall exterior enclosure of the diffuser, encapsulating both the water chamber and vial receiving areas 38. The inner cover 28 specifically covers the water chamber, featuring a domed wall portion that helps direct the generated mist toward the vent opening. The essential oil diffuser 10 is configured to store multiple essential oils and dispense precise amounts to create customized aromatic blends while enabling remote operation and monitoring.

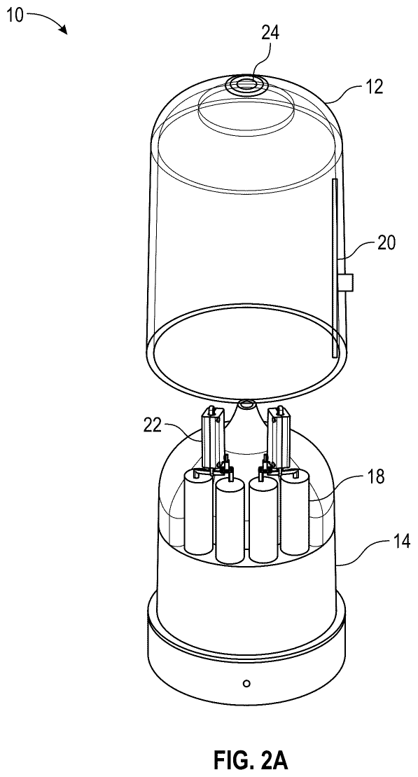

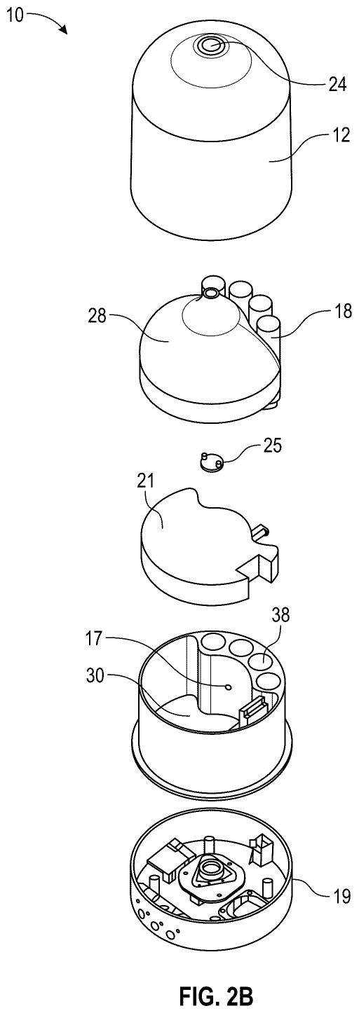

FIG. 2A illustrates an exploded view of the essential oil diffuser 10, in accordance with one exemplary embodiment of the present subject matter. The top housing 12 comprises a cylindrical shape with a vent opening 24 at the top, and an open bottom configured to mount on the bottom housing 14. The top housing 12 includes at least one actuator 22 positioned to engage with at least one vial when received in a respective vial receiving area. Electrical strips 20 are fitted on the inner side of the top housing 12 and configured to supply current to the at least one actuator 22. The at least one actuator 22 is configured to apply a downward force on the engaged vial to dispense essential oil through the oil dispensing port into the water chamber. In one embodiment, the at least one actuator 22 includes a first extension and a second extension configured to independently engage with two vials when received in respective vial receiving areas.

In this exemplary embodiment, the electrical connectivity between actuators 22 and electrical strips 20 is implemented through a reliable contact system. The electrical strips 20 are arranged along the inner circumference of the top housing 12 in a pattern corresponding to the actuator positions. Each actuator 22 includes dedicated electrical contact points that align with and make secure connection to these strips when the top housing 12 is properly mounted on the bottom housing 14. The electrical strips 20 comprise conductive material, such as copper or brass, and are configured in parallel circuits to ensure consistent power delivery to each actuator 22. Power is transmitted from the main control board in the base through wirings 44 that connect to the electrical strips 20. These wirings 44 are routed along the inner wall of the top housing 12 and secured with wire guides to prevent interference with the actuator movement. Each electrical strip 20 includes a positive and negative terminal to complete the circuit for actuator operation. When the top housing 12 is placed on the bottom housing 14, spring-loaded contacts in the electrical strips 20 ensure positive engagement with the actuator terminals, maintaining reliable electrical connection during operation. This arrangement enables independent control of each actuator 22 while providing easy disassembly for maintenance. The electrical strips 20 connect with the bottom housing 14 through magnetic connection that allows currents to travel from the upper housing to the base circuitry.

FIG. 2B shows an exploded view of the bottom housing's internal configuration. The top housing 12 comprises a cylindrical shape with a vent opening 24 at the top. The inner cover 28 with vials 18 attached to it in the inverted orientation. The bottom housing includes a water chamber 30 centrally positioned and formed by upstanding cylindrical walls. A plurality of vial receiving areas 38 are configured to receive the vials 18. A transducer (not shown) is mounted in the base of the water chamber 30 for generating mist from the water-oil mixture. The water chamber includes a removable floor 21 that fits securely into the base of the chamber and connects directly to the transducer. Oil dispensing ports 17 are strategically positioned around the periphery of the water chamber 30, with each port aligned with a corresponding vial receiving area to enable precise dispensing of essential oils into the water chamber. A sealed compartment 19 is provided within the base portion for housing electronic components including the controller, wireless communication modules, and power management systems. This compartment 19 is designed to keep the electronic components separate from the water chamber while maintaining easy access for maintenance. The compartment's location and sealing ensure protection of the electronic components from moisture while enabling efficient control of the diffuser's operations. A mounting element 25 is configured to mount the floor 21 into the water chamber 30. The mounting element 25 serves to disperse the mist and allow it to billow inside the inner cover 28 so mist comes out of vent opening 24 more evenly over time.

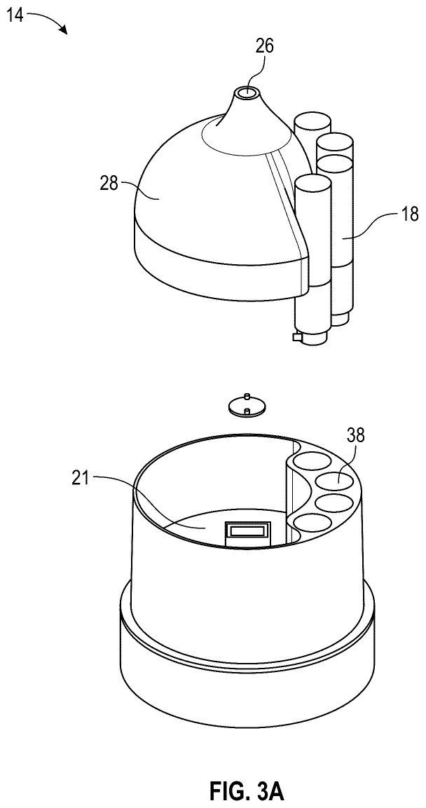

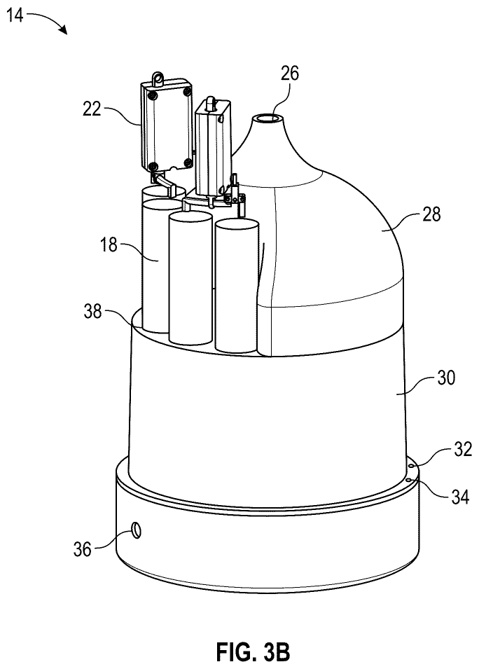

Now referring to FIG. 3A illustrates the exploded view of the bottom housing 14 features to accommodate vials 18 in an inverted orientation. The bottom housing 30 further comprises an inner cover 28. The inner cover 28 is a domed wall portion. The domed wall portion extends upward from the upstanding cylindrical walls 30 and converges toward the opening 26, with an interior surface configured to direct the generated mist from the water chamber toward the opening 26. A plurality of vial receiving areas 38 are configured to receive the vials 18. Each vial 18 is inserted upside-down into its respective vial receiving area, positioning the dispensing mechanism aligned with the oil dispensing ports 17 that lead into the water chamber. The water chamber includes a removable floor 21 that fits securely into the base of the chamber and connects directly to the transducer. The inverted orientation of the vials 18 enables gravity-assisted dispensing when activated either manually or electronically, ensuring consistent and precise delivery of essential oils into the water chamber. The floor's 21 connection to the transducer creates an integrated assembly that enhances the diffuser's mist generation capabilities while maintaining serviceable access to all components.

FIG. 3B illustrates a perspective view of the bottom housing 14, in accordance with one exemplary embodiment of the present subject matter. The bottom housing 14 includes a base having upstanding cylindrical walls forming a water chamber 30. The bottom housing 14 includes a transducer disposed in the water chamber and configured to generate a mist from a mixture of essential oil and water.

The bottom housing 14 comprises a plurality of vial receiving areas 38. In one embodiment, the plurality of vial receiving areas 38 comprises four vial receiving areas arranged in the bottom housing 14. Each vial receiving area 38 is configured to receive a vial 18 containing an essential oil in an inverted orientation. Each vial receiving area 38 comprises an oil dispensing port 17 opening into the water chamber.

Each vial 18 is configured to store essential oil and is calibrated to dispense the essential oil in increments of a predetermined volume. The vials 18 are removable from the vial receiving areas 38, interchangeable with other vials containing different essential oils, and reusable with a pump nozzle head for topical application.

In one embodiment, each vial is configured to be: (i) removable from the vial receiving area for refilling with the same or different essential oils; (ii) interchangeable with other vials containing different essential oils to allow users to swap between various therapeutic benefits (e.g., from relaxation blends to energizing blends); and (iii) reusable with a pump nozzle head for topical application when traveling. The vials feature a refillable design with a secure sealing mechanism that maintains the integrity of the stored essential oils during use and transport. When a vial's essential oil level becomes low, the user can simply remove it from the diffuser, refill it with their chosen essential oil, and reinsert it into the vial receiving area. This refillable capability provides cost-effective long-term use while enabling users to easily change between different essential oils based on their desired aromatherapy benefits. The vial's versatile design ensures reliable dispensing operation whether used with the original essential oil or refilled with a different variety.

The upper housing 12 includes at least one actuator 22 positioned to engage with each vial 18. In one embodiment, each actuator 22 includes a first extension and a second extension configured to independently engage with two vials 18 when received in respective vial receiving areas 38. The at least one actuator 22 are configured to apply different levels of force to dispense precise predetermined volumes of essential oil. In one exemplary embodiment, the at least one actuator is configured to provide controlled dispensing through multiple discrete actuations. Each actuation of the actuator creates a single dispensing operation that releases a precise, predetermined volume of essential oil (for example, one actuation dispenses 0.06 ml of oil). When executing a blend recipe that requires multiple drops from a single oil, the actuator performs the corresponding number of actuations on that vial (for example, 3 actuations to dispense 0.18 ml).

The positive and negative terminals 32, 34 connect to the electrical strips 20 in the top housing to supply power to the at least one actuator 22 when the top housing is mounted. The bottom housing 30 further comprises an inner cover 28. The inner cover 28 is a domed wall portion. The domed wall portion extends upward from the upstanding cylindrical walls 30 and converges toward the opening 26, with an interior surface configured to direct the generated mist from the water chamber toward the opening 26. The domed shape enhances the flow of mist toward the vent opening 24 while preventing condensation from dripping back into the water chamber.

A power button 36 is provided on the bottom housing 14 serving multiple functions in the operation of the essential oil diffuser 10. The power button 36 can be used for basic power control to turn the device on and off, as well as manual activation of the at least one actuator 22 for dispensing essential oils from the vials when pressed in specific patterns. In advanced operation modes, the power button 36 can be used to initiate predetermined blend recipes and control wireless communication features. These power button functions can also be accessed remotely through an electronic device connected via the wireless network, providing users with convenient control options for both manual and automated operation of the essential oil diffuser.

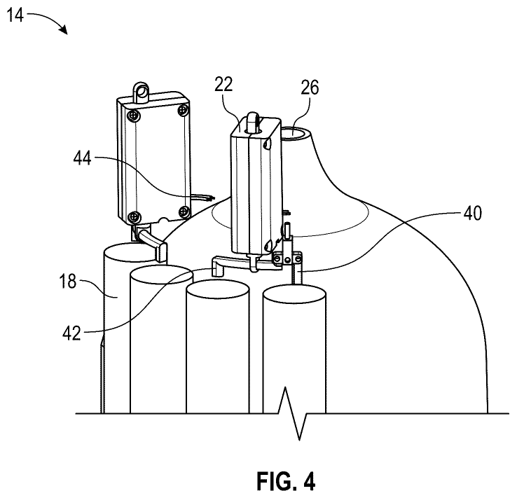

FIG. 4 illustrates a perspective view of the bottom housing 14 showing engagement between the at least one actuator 22 and one or more vials 18. The one or more vials 18 are configured to be received in an inverted orientation in the vial receiving areas 38 of the bottom housing 14, with their dispensing ends facing downward toward the water chamber.

In one embodiment, each vial 18 has a dedicated the at least one actuator 22 for independent control. The power is transmitted to the at least one actuator 22 through wirings 44. The wirings 44 are connected to the electrical strips 20. In another embodiment, each actuator 22 includes a first extension 40 and second extension 42 configured to independently engage with two vials 18 to optimize operation while maintaining precise dispensing control.

The at least one actuator 22 applies downward force on the vial 18, compressing the internal plunger assembly comprising a piston component and spring. This creates precise displacement forcing measured quantities of essential oil past the ball check valve through the oil dispensing port 17 into the water chamber. The spring return to their initial positions upon force release, while the ball component prevents fluid backflow.

After dispensing, the transducer generates a mist from the oil-water mixture which is directed through the vent opening 24. This inverted vial configuration with precision actuation ensures reliable dispensing while maintaining the integrity of the stored contents.

The bottom housing 14 includes a water chamber formed by upstanding cylindrical walls 30 and a base portion. The base portion contains a controller, a transceiver electronically connected to the controller for wireless network communication, a power source, and a memory storing executable instructions

The controller is electronically connected to the at least one actuator 22 through electrical strips 20 in the top housing 12. The controller activates the at least one actuator 22 according to predetermined schedules and blend recipes received from an electronic device via a transceiver over a wireless network. The controller can monitor essential oil levels in each vial 18 and transmits notifications when levels fall below predetermined thresholds.

The controller is configured to receive blend recipes comprising instructions for dispensing different amounts of essential oils from multiple vials 18. In one exemplary embodiment, the at least one actuator is configured to provide controlled dispensing through multiple discrete actuations. Each actuation of the actuator creates a single dispensing operation that releases a precise, predetermined volume of essential oil.

The controller maintains usage logs for each vial 18, calculates estimated remaining usage volume based on predetermined volume capacity, and automatically transmits reordering notifications when usage volume falls below thresholds. Additionally, the controller stores usage patterns of different essential oil combinations to generate and transmit recommended blend recipes to the connected electronic device. The controller is configured to activate the actuator according to a predetermined schedule received from the electronic device.

The vials 18 are removable from the vial receiving areas 38, interchangeable with other vials containing different essential oils, and reusable with a pump nozzle head for topical application. Each vial 18 is configured to store and dispense essential oil in calibrated increments of a predetermined volume.

In one exemplary embodiment, the actuator assembly is configured to provide precise volume control through multiple discrete actuations. Each actuation of the actuator mechanism creates a single dispensing operation that releases a consistent, predetermined volume of essential oil (for example, 0.06 ml per actuation) through the interaction between the actuator and the vial's internal plunger assembly. When executing a blend recipe requiring different volumes of oils, the controller activates the actuator to perform different numbers of actuations on selected vials. For example, to dispense 0.18 ml from a first vial, the actuator performs three sequential actuations on that vial, while dispensing 0.06 ml from a second vial requires just one actuation. The actuator's mechanical design, coupled with the vial's calibrated internal components including the spring-loaded plunger and check valve, ensures that each actuation consistently dispenses the same measured volume regardless of the vial's fill level or the number of actuations performed. The controller could precisely track these actuations to maintain accurate volume control according to blend recipe requirements, enabling the creation of complex oil combinations while maintaining operational simplicity and reliability. Each actuation involves a complete cycle of the actuator engaging the vial's plunger assembly, compressing it to dispense the oil, and returning to its rest position, ready for subsequent actuations if needed for the recipe.

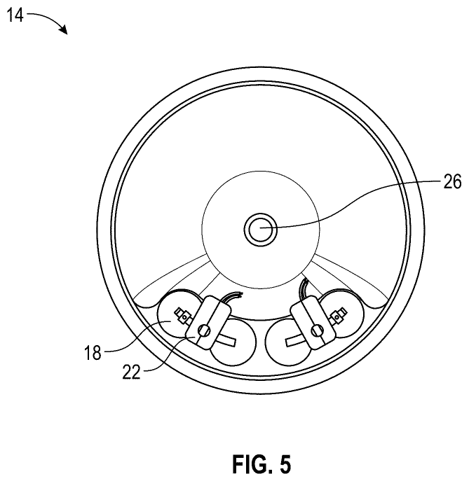

FIG. 5 illustrates a top view of the bottom housing 14, in accordance with one exemplary embodiment of the present subject matter.

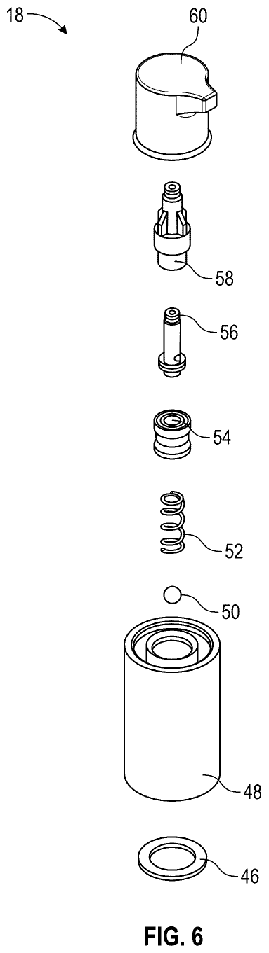

FIG. 6 illustrates an exploded view of vial 18, in accordance with one exemplary embodiment of the present subject matter. Each vial 18 comprises a glass bottle for storing the essential oil, a cylindrical casing 48, a cap 60, a plunger assembly (including plunger top part 58, piston component 56, and plunger bottom part 54), a sealant 46, a spring 52, and a ball 50.

The cylindrical casing 48 forms the main body having an open first end for component assembly and a second end with sealing features 46 for fluid-tight engagement with the oil dispensing port 17. The cylindrical casing includes internal guide ribs and seats for proper component alignment. The cap 60 includes a dome-shaped upper surface and cylindrical side walls providing sealed closure while guiding the at least one actuator 22 engagement. The cap 60 is aligned with the oil dispensing port 17. The at least one actuator 22 push the bottom of the vial to dispense essential oil out of the cap (60) and into the water through the oil dispensing port 17.

The plunger top part 58 includes radially extending guide features maintaining aligned vertical movement. The piston component 56 has circumferential sealing features creating an airtight seal against the casing inner wall. The plunger bottom part 54 contains internal fluid channels directing essential oil flow during dispensing.

The spring 52 is disposed below the plunger assembly, engineered to provide consistent return force for resetting the mechanism. The ball 50 positioned below the spring functions as a one-way valve controlling fluid flow direction.

During operation, the at least one actuator 22 applies downward force on bottom of the glass bottle which then depress the plunger assembly. The piston creates precise oil displacement through the fluid channels while compressing the spring. Oil pressure unseats the ball allowing measured release. When force is removed, spring tension returns components to their initial positions while the ball prevents backflow. This integrated mechanism enables dispensing of precise oil volumes for blend recipes.

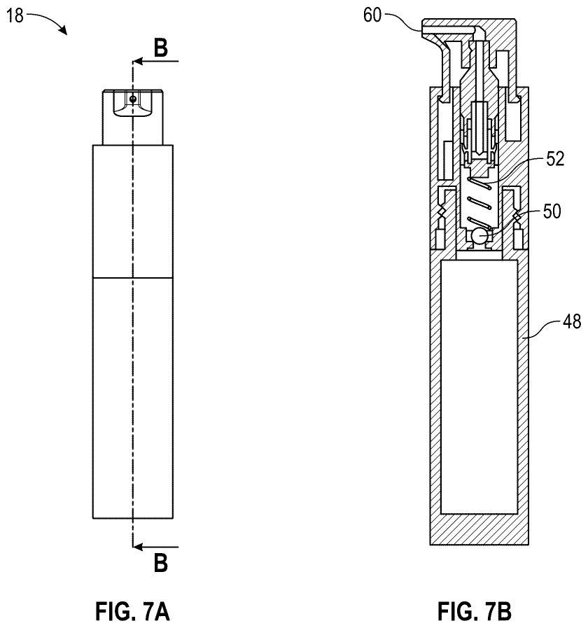

FIGS. 7A and 7B illustrate front and cross-sectional views of the vial 18, in accordance with one exemplary embodiment of the present subject matter. The cross-sectional view of FIG. 7B shows the coaxial arrangement of internal components within the cylindrical casing 48. The plunger top part 58, piston component 56, and plunger bottom part 54 are aligned for linear movement along the vial's longitudinal axis. In the assembled state, the spring 52 maintains partial compression between the plunger bottom part 54 and ball 50.

Referring back to FIG. 2, top housing 12 includes a vent opening 24 at the top. In one example, bottom housing 14 includes a battery (not shown) for operating transducer (not shown), a controller (not shown), a transceiver (not shown). In another example, bottom housing 14 includes a cable (not shown) connecting to a power source (not shown) for operating transducer.

In use, a user places vials 18 in vial receiving areas 38 as shown in FIG. 3. As specified above, each vial 18 contains a different essential oil with varied composition. The user fills water in water chamber and places top housing 12 over bottom housing 14. In one exemplary embodiment, when needed, the user actuates vial 18 by pressing at the top to dispense essential oil into water chamber 20. In one example, the user physically pushes at the top of vial 18 to eject one measured drop of essential oil directly into water chamber. Here, the essential oil from vial 18 enters water chamber via oil dispensing port and mixes with water. Transducer vibrates causing the essential oil and the water to evaporate to mist particles. In other words, transducer vibrates such that small droplets of water, and the essential oil are released into the air as a cool mist. The mist particles are released from the vent opening 24. In accordance with the present subject matter, the user actuates one or more vials 18 to dispense the essential oil into water chamber, which releases the mist of water and essential oil into air via vent opening 24 to deliver aromatic or therapeutic properties of the oils used. Once the essential oil levels drop or are finished in vial 18, the user may top up, refill, or completely change the type of essential oil in vial 18.

In another embodiment, each vial is actuated using buttons provided on one of bottom housing 12 and top housing 14. FIG. 3 shows the feature of bottom housing 12 having buttons 36. In one example, each vial 18 is configured to be operated by actuating of a single button i.e., four buttons might be provided to operate four vials 18 . . . . At the bottom, vial 18 includes a one-way pump valve for dispensing essential oil stored in vial 18. In operation, when button 36 is pressed, an electronic signal is sent to the at least one actuator 22. The at least one actuator 22 moves down while being around the walls of vial 18 causing essential oil to flow down. The essential oil is made to flow into water chamber. After dispensing the essential oil into water chamber, transducer vibrates causing the essential oil and the water to evaporate into mist particles. Subsequently, the mist particles are released into the air via the vent opening 24.

In the preferred embodiment, the essential oil diffuser 10 includes intelligent control features for creating customized essential oil blends. The controller receives blend recipes via the transceiver from an electronic device, with each recipe comprising instructions for dispensing number of drops of different essential oils from selected vials. The at least one actuator 22 is configured perform actuation to dispense a predetermined volume of essential oil, wherein each actuation dispenses the predetermined volume of essential oil.

For each vial, the controller logs dispensing operations, tracks remaining volume, and calculates estimated usage volume based on predetermined vial capacity. When remaining usage volume falls below a threshold, the controller automatically transmits notifications to the connected electronic device.

In one embodiment, the controller analyses usage patterns of different oil combinations to generate recommended blend recipes. These personalized recommendations are transmitted to the electronic device, enabling users to discover new beneficial combinations based on their preferences and usage history. The controller stores successful blend recipes in memory for subsequent use. This allows users to consistently recreate preferred oil combinations while maintaining precise control over the aromatic properties of the generated mist. The wireless connectivity enables remote operation and monitoring. Users can initiate blend recipes, check oil levels, and receive notifications through the electronic device. The transceiver maintains reliable communication over the wireless network, ensuring responsive control and timely status updates. Through the combination of precise actuator control, intelligent usage tracking, and wireless connectivity, the essential oil diffuser 10 provides an automated and customizable aromatherapy experience while ensuring optimal essential oil utilization.

When a vial 18 installed in the vial receiving area 38 in an inverted orientation, the at least one actuator 22 engages with the bottom side of the vial 18. Upon actuation, downward force drives the plunger assembly causing the piston 56 to create precise displacement. This movement compresses spring 52 while forcing a measured volume of essential oil past ball 50 through the oil dispensing port 17. When the at least one actuator force releases, spring tension returns components to their starting positions as the ball prevents fluid backflow, maintaining dispensing accuracy.

The controller determines remaining oil levels in each vial through precision monitoring of actuator operations. Each actuation dispenses a calibrated volume (0.06 ml) of essential oil. The controller maintains a running tally by subtracting dispensed volumes from the initial 10 ml vial capacity. Integrated sensors in the actuator mechanism detect resistance changes that indicate when a vial approaches empty, triggering low-level notifications.

The essential oil diffuser implements industry-standard wireless protocols including Wi-Fi (IEEE 802.11 b/g/n) and Bluetooth Low Energy 5.0 for communication with electronic devices. The transceiver utilizes encrypted data transmission with AES-256 security. The wireless stack supports TCP/IP for network connectivity and MQTT protocol for efficient message exchange, enabling real-time control and monitoring through the companion mobile application.

The actuator mechanism employs actuation control through precision linear actuation. The actuation creates 0.06 ml displacement by compressing the spring component to its first detent position. The second actuation achieves 0.06 ml displacement to a second detent. The actuator's electronic driver circuit maintains consistent force application across the full range of vial fill levels.

In one exemplary embodiment, an essential oil diffuser comprising a bottom housing and a top housing. The bottom housing comprising a base having upstanding cylindrical walls forming a water chamber, a plurality of vial receiving areas, a transducer disposed in the water chamber and a controller. Each vial receiving area is configured to receive a vial containing an essential oil in an inverted orientation. Each vial receiving area comprises an oil dispensing port opening into the water chamber. The top housing is mounted on the bottom housing. The top housing comprises the at least one actuator positioned to engage with at least one vial when received in a respective vial receiving area. The at least one actuator is configured to apply a downward force on the engaged vial to dispense essential oil through the oil dispensing port into the water chamber. The transducer is configured to generate a mist from a mixture of the essential oil and water in the water chamber. A vent opening configured to release the generated mist. The controller electronically is connected to the at least one actuator and the transducer. A transceiver electronically connected to the controller and configured to communicate with an electronic device over a wireless network. A memory storing executable instructions that, when executed by the controller, cause the controller to perform a set of steps. The set of steps includes receiving blend recipes from the electronic device, activating the at least one actuator according to the blend recipes to dispense customized mixtures of essential oils in the water chamber, and monitoring amounts of essential oils remaining in the vials and transmit notifications to the electronic device. The controller comprises electronic components and circuitry for processing instructions and controlling essential oil diffuser operations.

The controller includes a microprocessor executing the stored instructions, memory modules storing operational data, and interface circuitry connecting to actuator, transducer, and transceiver.

The memory includes non-volatile storage retaining data when powered off and volatile memory for temporary operation storage. The non-volatile memory stores blend recipes, usage logs, and control parameters. The volatile memory handles real-time processing of dispensing operations and sensor readings.

The transceiver enables bi-directional wireless communication using standard protocols. It comprises radio frequency circuits for transmitting notifications and receiving instructions from electronic devices. The wireless connectivity allows remote operation through mobile applications or other networked interfaces.

In another embodiment, a method of operating an essential oil diffuser is disclosed. The method comprising various steps.

In first step, a plurality of vials is received in respective vial receiving areas of a bottom housing in an inverted orientation. Each vial is containing an essential oil. Each vial receiving area comprises an oil dispensing port opening into a water chamber formed by upstanding cylindrical walls of the bottom housing.

In the next step, water is filled in the water chamber to a predetermined level.

In use, the essential oil diffuser 10 provides users with two convenient methods for dispensing essential oils. In addition to the electronic actuation system described above, users may choose to manually dispense oils directly into the water chamber. For manual operation, the user can apply physical pressure with their finger to the top of any vial 18 installed in the vial receiving areas 38. Each downward press on a vial 18 activates its internal plunger mechanism to dispense a precise amount of essential oil (for example, 0.06 ml per press) through the oil dispensing port into the water chamber. The vial's calibrated internal spring and check valve system ensures consistent dispensing volume whether activated electronically or manually.

In the next step, a top housing is mounted on the bottom housing. The top housing includes at least one actuator positioned above at least one of the received vials.

In the next step, the at least one actuator is activated to apply a downward force on the at least one received vial, thereby causing essential oil to be dispensed through the oil dispensing port into the water chamber.

In the last step, a transducer is operated to generate a mist from a mixture of the dispensed essential oil and the water and releasing the generated mist through a vent opening in the top housing.

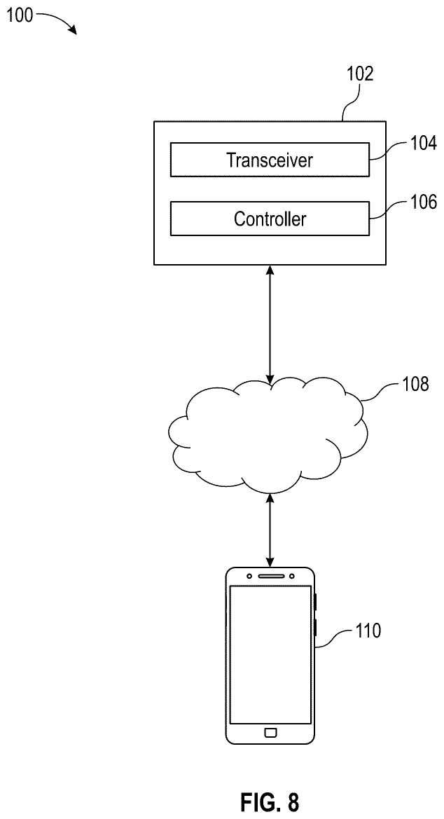

FIG. 8 shows an environment 100 of essential oil diffuser 10 communicatively connected to an electronic device 110 via a network 108. Here, electronic device 110 includes one of a mobile device, a personal digital assistant, a laptop computer, a tablet computer, a desktop computer, a voice activated device, a smart watch, etc. Network 108 includes a wireless network, a wired network or a combination thereof. Network 108 can be implemented as one of the different types of networks, such as intranet, local area network (LAN), wide area network (WAN), the internet, and the like. Network 108 implements as a dedicated network or a shared network. The shared network represents an association of the different types of networks that use a variety of protocols, for example, Hypertext Transfer Protocol (HTTP), Transmission Control Protocol/Internet Protocol (TCP/IP), Wireless Application Protocol (WAP), and the like, to communicate with one another. Further the network 108 includes a variety of network devices, including routers, bridges, servers, computing devices, storage devices, and the like.

In accordance with the present embodiment, essential oil diffuser 10 includes a transceiver 104. Transceiver 104 transmits or receives software instructions over network 108 with electronic device 110. Transceiver 104 utilises any one of a number of well-known transfer protocols to transmit or receive the software instructions over network 108. Further, essential oil diffuser 10 includes a controller 106 electronically connected to transceiver 104. In one example, vial 18 is operated by the at least one actuator 22 to dispense the essential oil, as explained above. The user uses an application on electronic device 110 to operate vial 18 to dispense the essential oil into water chamber, cause the essential oil and the water to evaporate to mist particles and release it into the air. In one example, electronic device 110 presents options to select vials 18 to dispense the essential oil individually and separately from one another. In another example, electronic device 110 presents options to select one or more vials 18 and dispense the essential oil from one or more vials 18 simultaneously into water chamber. A person skilled in the art understands that electronic device 110 allows the user to control i.e., start/stop of essential oil diffuser 10 and its settings (including lights and time to switch ON/OFF) remotely. For example, electronic device 110 can be used to configure settings to instruct the essential oil diffuser 10 start/stop operation at a predefined time on every day, say at 7 AM for a period of 30 minutes.

In one embodiment, controller 106 obtains information about the amount/quantity of essential oil remaining in the vial 18. When the quantity is low, then controller 106 sends signal to electronic device 110. In one example, electronic device 110 displays an alert or notification about the amount/quantity of essential oil remaining in the vial 18. In some instances, electronic device 110 automatically places an order on an electronic commerce marketplace for purchasing a new vial or refill to replenish vial 18 with essential oil.

In one example, the application in electronic device 110 provides information to the user about the contents of each vial 18 to educate the users on essential oils and their purpose in diffusing. For example, the application presents information of the essential oil such as a recipe for the users with the stored essential oils to diffuse for the purpose of sleep, focus, relaxation, etc. In another example, the application provides information to create a combination of essential oils (i.e., recipes) to diffuse for different purposes—better sleep, easier waking up or focused studying, etc.

In addition, the presently disclosed essential oil diffuser offers enhanced connectivity through the application for more than just on/off functionality when compared with known devices. Further, the presently disclosed essential oil diffuser presents personalized start times (e.g., for morning wake up strategies, etc) and recipe suggestion as the application recognizes the contents of each oil storage vial. In some instances, the application can even suggest recipes (and corresponding oils needed) based on health benefits marked as desired by the user.

Further, the essential oil diffuser presents interchangeability of oil vials (instead of just pouring oil into a reservoir and waiting to use it all up before changing to another oil) to allow the user to store and use different oils swiftly to allow them to change what health benefits they want to address at the time of use (i.e., change from oils for sleep to oils for concentration to oils for stress management and more). Additionally, the essential oil diffuser offers portability of oil storage vials by simply unplugging them from one diffuser and a) plugging them into another diffuser of the same brand or b) capping them with the provided pump nozzle head to take on the road and allow use of their oils topically while traveling. When the user gets home, they can simply re-plug the oil storage vial back into their desired diffuser to diffuse into the air once again.

The present subject matter has been described in particular detail with respect to various possible embodiments, and those of skill in the art will appreciate that the subject matter may be practiced in other embodiments. First, the particular naming of the components, capitalization of terms, the attributes, data structures, or any other programming or structural aspect is not mandatory or significant, and the mechanisms that implement the subject matter or its features may have different names, formats, or protocols. Further, the system may be implemented via a combination of hardware and software, as described, or entirely in hardware elements. Also, the particular division of functionality between the various system components described herein is merely exemplary, and not mandatory; functions performed by a single system component may instead be performed by multiple components, and functions performed by multiple components may instead be performed by a single component.

Some portions of the above description present the features of the present subject matter in terms of algorithms and symbolic representations of operations on information. These algorithmic descriptions and representations are the means used by those skilled in the data processing arts to most effectively convey the substance of their work to others skilled in the art. These operations, while described functionally or logically, are understood to be implemented by computer programs.

Further, certain aspects of the present subject matter include process steps and instructions described herein in the form of an algorithm. It should be noted that the process steps and instructions of the present subject matter could be embodied in software, firmware or hardware, and when embodied in software, could be downloaded to reside on and be operated from different platforms used by real time network operating systems.

The algorithms and operations presented herein are not inherently related to any particular computer or other apparatus. Various general-purpose systems may also be used with programs in accordance with the teachings herein, or it may prove convenient to construct more specialized apparatus to perform the required method steps. The required structure for a variety of these systems will be apparent to those of skill in the, along with equivalent variations. In addition, the present subject matter is not described with reference to any particular programming language. It is appreciated that a variety of programming languages may be used to implement the teachings of the present subject matter as described herein, and any references to specific languages are provided for disclosure of enablement and best mode of the present subject matter.

It should be understood that components shown in figures are provided for illustrative purposes only and should not be construed in a limited sense. A person skilled in the art will appreciate alternate components that might be used to implement the embodiments of the present subject matter, and such implementations will be within the scope of the present subject matter.

While preferred embodiments have been described above and illustrated in the accompanying drawings, it will be evident to those skilled in the art that modifications may be made without departing from this subject matter. Such modifications are considered as possible variants within the scope of the subject matter.

What is claimed is:

1. An essential oil diffuser comprising:
a bottom housing comprising:
a base having upstanding cylindrical walls forming a water chamber;
a plurality of vial receiving areas, each vial receiving area configured to receive a vial of a plurality of vials, each vial containing an essential oil in an inverted orientation, and wherein each vial receiving area comprises an oil dispensing port opening into the water chamber; and
a transducer disposed in the water chamber;
a top housing mounted on the bottom housing, wherein the top housing comprises at least one actuator positioned to engage with at least one vial when received in a respective vial receiving area, and wherein the at least one actuator configured to apply a force on the at least one vial to dispense essential oil through the oil dispensing port into the water chamber; and
a vent opening configured to release the generated mist,
wherein the transducer is configured to generate a mist from a mixture of the essential oil and water in the water chamber.

2. The essential oil diffuser of claim 1, wherein each vial comprises a glass bottle for storing the essential oil, a cylindrical casing, a plunger assembly movably disposed within the cylindrical casing, a spring and a ball positioned below the spring, and wherein the ball functions as a check valve to enable unidirectional fluid flow during dispensing operations.

3. The essential oil diffuser of claim 1, wherein the at least one actuator comprises a first extension and a second extension configured to independently engage with two vials of the plurality of vials when received in respective vial receiving areas.

4. The essential oil diffuser of claim 1, further comprising:
a controller; and
a transceiver electronically connected to the controller,
wherein the transceiver is configured to communicate with an electronic device over a network to receive control signals for operating the at least one actuator.

5. The essential oil diffuser of claim 4, wherein the controller is configured to:
monitor an amount of essential oil remaining in each vial; and
transmit, via the transceiver, a notification to the electronic device when the amount of essential oil in any vial falls below a predetermined threshold.

6. The essential oil diffuser of claim 4, wherein the controller is configured to activate the actuator according to a predetermined schedule received from the electronic device.

7. The essential oil diffuser of claim 1, wherein the plurality of vial receiving areas comprises four vial receiving areas arranged in the bottom housing.

8. The essential oil diffuser of claim 1, wherein each vial of the plurality of vials is configured to store the essential oil and is calibrated to dispense the essential oil in increments of a predetermined volume.

9. The essential oil diffuser of claim 1, further comprises electrical strips disposed on an inner side of the top housing and configured to supply current to the at least one actuator.

10. The essential oil diffuser of claim 1, wherein each vial is configured to be:
removable from the vial receiving area;
refillable with the same or different essential oils when removed;
interchangeable with other vials containing different essential oils; and
reusable with a pump nozzle head for topical application.

11. The essential oil diffuser of claim 1, wherein the bottom housing comprises an inner cover, wherein the inner cover is a domed wall portion extending upward from the upstanding cylindrical walls and converging toward the vent opening of the top housing, and wherein the domed wall portion includes an interior surface configured to direct the generated mist from the water chamber toward the vent opening.

12. The essential oil diffuser of claim 4, wherein the controller is configured to:
receive, via the transceiver, a blend recipe comprising instructions for dispensing at least one essential oil from at least one vial; and
activate the at least one actuator according to the blend recipe to dispense a mixture of essential oils in the water chamber.

13. The essential oil diffuser of claim 12, wherein the at least one actuator is configured to:
perform actuation to dispense a predetermined volume of essential oil, wherein each actuation dispenses the predetermined volume of essential oil.

14. The essential oil diffuser of claim 12, wherein the controller is further configured to:
store usage patterns of different essential oil combinations;
store the blend recipe for subsequent use; and
generate and transmit recommended blend recipes to the electronic device based on the stored usage patterns.

15. The essential oil diffuser of claim 14, wherein the controller is further configured to:
maintain a usage log for each vial received in the vial receiving areas;
calculate an estimated remaining usage volume for each vial based on the respective usage log and a predetermined volume capacity; and
automatically transmit, via the transceiver to the electronic device, a reordering notification when the estimated remaining usage volume falls below a predetermined threshold.

16. An essential oil diffuser, comprising:
a bottom housing comprising:
a base having upstanding cylindrical walls forming a water chamber; and
a plurality of vial receiving areas, each vial receiving area configured to receive a vial containing an essential oil in an inverted orientation, wherein each vial receiving area comprises an oil dispensing port opening into the water chamber; and a transducer disposed in the water chamber;
a top housing mounted on the bottom housing, wherein the top housing comprises at least one actuator positioned to engage with at least one vial when received in a respective vial receiving area, the at least one actuator configured to apply a downward force on the engaged vial to dispense essential oil through the oil dispensing port into the water chamber, wherein the transducer is configured to generate a mist from a mixture of the essential oil and water in the water chamber,
a vent opening configured to release the generated mist;
a controller electronically connected to the at least one actuator and the transducer;
a transceiver electronically connected to the controller and configured to communicate with an electronic device over a wireless network; and
a memory storing executable instructions that, when executed by the controller, cause the controller to:
receive blend recipes from the electronic device;
activate the at least one actuator according to the blend recipes to create customized mixtures of essential oils in the water chamber; and
monitor amounts of essential oils remaining in the vials and transmit notifications to the electronic device.

17. A method of providing an essential oil diffuser, the method comprising the steps of:
receiving a plurality of vials in respective vial receiving areas of a bottom housing in an inverted orientation, each vial containing an essential oil, wherein each vial receiving area comprises an oil dispensing port opening into a water chamber formed by upstanding cylindrical walls of the bottom housing;
filling the water chamber with water to a predetermined level;
mounting a top housing on the bottom housing, wherein the top housing includes at least one actuator positioned above at least one of the received vials;
activating the at least one actuator to apply a downward force on the at least one received vial, thereby causing essential oil to be dispensed through the oil dispensing port into the water chamber; and
operating a transducer disposed in the water chamber to generate a mist from a mixture of the dispensed essential oil and the water; and releasing the generated mist through a vent opening in the top housing.

18. The method of claim 17, further comprising:
monitoring an amount of essential oil remaining in each vial; and
transmitting a notification to an electronic device when the amount of essential oil in the vial falls below a predetermined threshold.
